(19) United States
(12) Patent Application Publication
(10) Pub. No.: US 2018/0169092 A1
(43) Pub. Date: Jun. 21, 2018

(54) PHARMACEUTICAL FORMULATIONS FOR TREATING GLAUCOMA AND METHODS FOR FABRICATING AND USING THEREOF

(71) Applicant: Imprimis Pharmaceuticals, Inc., San Diego, CA (US)

(72) Inventors: Dennis Elias Saadeh, Irvine, CA (US); Mark L. Baum, San Diego, CA (US)

(21) Appl. No.: 15/832,169

(22) Filed: Dec. 5, 2017

Related U.S. Application Data
(60) Provisional application No. 62/434,942, filed on Dec. 15, 2016.

(51) Int. Cl. A61K 31/498; A61K 31/382; A61K 31/5377; A61K 31/216; A61K 47/34; A61K 47/38; A61K 9/00; A61K 45/06

(52) U.S. Cl. CPC A61K 31/498; A61K 31/382; A61K 31/5377; A61K 31/216; A61K 2300/00; A61K 47/38; A61K 9/0048; A61K 45/06; A61K 47/34

(57) ABSTRACT

Pharmaceutical compositions for treating or mitigating glaucoma are described, the compositions comprising several separate components for an improved effect. Methods for fabricating the compositions and using them are also described.

### CROSS-REFERENCE TO RELATED APPLICATION(S)

This application claims the benefit of priority under 35 U.S.C. § 119(e) of U.S. Provisional Application No. 62/434,942, filed Dec. 15, 2016, the entire content of which is incorporated herein by reference.

### FIELD OF THE INVENTION

The present invention relates generally to the field of ophthalmology, and more specifically to compositions and methods designed to treat or mitigate glaucoma, and to methods of preparing such compositions.

### BACKGROUND

A significant portion of the population worldwide suffers from glaucoma (including open- and close-angle varieties), which is a very common condition typically characterized by an increase in the eye pressure, which may cause damage to the optic nerve and eventually lead to vision loss, especially if not properly treated.

It is well known that glaucoma, particularly the open-angle kind, develops slowly over time, with no pain or other patient-discernable effects. Side vision may begin to decrease, followed by central vision resulting in blindness, if not treated. Vision loss from glaucoma, once it has occurred, is permanent and irreversible.

Current treatments include both invasive (i.e., surgery, laser treatment) and non-invasive (i.e., medication) types, and the latter typically include the use of topical eye drops based on prostaglandin analogs, on topical beta-adrenergic receptor antagonists, highly selective α-2-adrenergic agonists or less selective α-agonists, on parasympathomimetics, and on carbonic anhydrase inhibitors, all with the purpose of reducing intraocular pressure.

All such treatments, however, are of limited effectiveness in many patients due to the possibility of causing local and/or systemic side effects, poor patient tolerance in some cases, as well as poor compliance with the treatment regimen. Further complicating the non-invasive treatment protocol is the necessity of using, in some cases, not one but several separate medications in sequence.

In addition, many existing medications for the treatment of glaucoma are often perishable products with limited shelf life and limited stability, requiring the use of preservatives and/or stabilizers, for a prolonged shelf life and stability.

However, using such medications containing preservatives is undesirable in many cases as it can cause toxicity in the eye. Although most such cases of toxicity are cured with topical steroids, severe cases can lead to cornea transplantation and iris atrophy. Having alternative non-toxic epinephrine-based compositions and procedures utilizing them that are safer, but equally effective is, therefore, desirable.

Accordingly, there exists a need for better methods and compositions for treatment and/or mitigation of glaucoma. This patent specification discloses such pharmaceutical compositions that would achieve positive patient outcomes while being free of drawbacks and deficiencies of existing formulations, and methods of fabricating and administering the same.

### SUMMARY

According to one embodiment of the invention, a pharmaceutical composition for treating or mitigating glaucoma is provided. The composition comprises a therapeutically effective quantity of at least one pharmaceutically acceptable α-2-adrenergic agonist, at least one carbonic anhydrase inhibitor, at least one β-adrenergic antagonist, and at least one prostaglandin analog. In further embodiments, the composition may contain only two or three of such components. In yet further embodiments, the composition is free or at least essentially free of preservatives and/or stabilizers.

According to further embodiments of the invention, the pharmaceutical composition additionally comprises a quantity of at least one solubilizing and suspending agent and is formulated as a suspension.

According to other embodiments of the invention, a method for treating or mitigating glaucoma is provided. The method includes administering to a patient in need thereof any of the embodiments of the above-mentioned pharmaceutical composition.

### DETAILED DESCRIPTION

### A. Terms and Definitions

Unless specific definitions are provided, the nomenclatures utilized in connection with, and the laboratory procedures and techniques of analytical chemistry, synthetic organic and inorganic chemistry described herein, are those known in the art. Standard chemical symbols are used interchangeably with the full names represented by such symbols. Thus, for example, the terms “hydrogen” and “H” are understood to have identical meaning. Standard techniques may be used for chemical syntheses, chemical analyses, formulating compositions and testing them. The foregoing techniques and procedures can be generally performed according to conventional methods well known in the art.

It is to be understood that both the foregoing general description and the following detailed description are exemplary and explanatory only and are not restrictive of the invention claimed. As used herein, the use of the singular includes the plural unless specifically stated otherwise. The section headings used herein are for organizational purposes only and are not to be construed as limiting the subject matter described.

As used herein, “or” means “and/or” unless stated otherwise. Furthermore, use of the term “including” as well as other forms, such as “includes,” and “included,” is not limiting.

“About” as used herein means that a number referred to as “about” comprises the recited number plus or minus 1-10% of that recited number. For example, “about” 100 degrees can mean 95-105 degrees or as few as 99-101 degrees depending on the context. Whenever it appears herein, a numerical range such as “1 to 20” refers to each integer in the given range; i.e., meaning only 1, only 2, only 3, etc., up to and including only 20.

The term “pharmaceutical composition” is defined as a chemical or biological compound or substance, or a mixture or combination of two or more such compounds or substances, intended for use in the medical diagnosis, cure, treatment, or prevention of disease or pathology.

The term “glaucoma” refers to a group of eye conditions that damage the optic nerve, which is often caused by an abnormally high pressure in the eye.

The term “open-angle glaucoma” refers to the most common type of glaucoma, whereby there is a wide and open angle between the iris and cornea and the increased eye pressure is the result of the slow clogging of the drainage canals.

The term “α-2-adrenergic agonists” refers to a class of sympathomimetic agents that selectively stimulates a-adrenergic receptors (i.e., a group of proteins that sense molecules outside the cell and activate inside signal transduction pathways and cellular responses).

The term “carbonic anhydrase inhibitors” refers to a class of compounds that suppress the activity of carbonic anhydrase (i.e., the activity of enzymes that reversibly catalyze the interconversion of carbon dioxide and water to bicarbonate and protons).

The term “β-adrenergic antagonists” also known as β-blockers” refers to compounds that are capable of blocking the effects of the hormone epinephrine (adrenaline).

The term “prostaglandin analogs” refers to compounds that are capable of binding to a prostaglandin receptor (i.e., to G protein-coupled receptors that bind and are activated by prostaglandin).

The term “topical” refers to a medicament that is applied directly to a particular or specific place on or in the body of a patient, as opposed to being systemically administered.

The term “eye drops” refers to a medicated solution for the eyes that is applied in form of drops using, e.g., an eyedropper.

The terms “synergy” or “synergistic” refer to a combined result of the interaction of two or more compounds or kinds of medication, that, when used together, produce a combined effect greater than the sum of their separate effects.

The term “suspension” is defined for the purposes of the present application as a two-phase dispersion system having a first phase and a second phase. It is further specifically provided that dispersion systems having three, four or more phases are not within the meaning of “suspension” for the purposes of the instant application.

Furthermore, the above mentioned first phase of the suspension consists of a multitude of solid particles and is designated and defined as the dispersed phase, and the above mentioned second phase of the suspension is a liquid and is designated and defined as the dispersion medium, or, interchangeably and synonymously, the continuous phase.

Furthermore, the above mentioned dispersed phase is dispersed in the above mentioned dispersion medium, and the term “dispersed” is defined as meaning that the dispersed phase is statistically evenly distributed throughout the entire volume of the suspension, with no statistically meaningful deviations in the concentrations of the dispersed phase in different portions of the suspension.

The term “solubilizing agent” for the purposes of the instant application refers broadly to chemical compounds that improve the process of incorporating the solubilizate (i.e., active components described herein) into micelles; in other words the presence of a solubilizing agent makes the process of solubilization faster, easier, and/or more complete compared with compositions without it.

The term “suspending agent” for the purposes of the instant application refers broadly to chemical compounds that help active pharmaceutical ingredients stay suspended in the formulation and prevents and/or reduces the phase separation of two-phase dispersion systems described herein.

The term “preservative” for the purposes of the present invention refers to a chemical substance that is added to a pharmaceutical composition to prevent the pharmaceutical composition from deterioration, decomposition or degradation or to substantially reduce or decelerate the degree and/or the speed of such deterioration, decomposition or degradation.

Accordingly, “preservative-free” means a pharmaceutical composition that does not include a preservative or includes not more than a trace amount of a preservative. Thus, the pharmaceutical composition can be completely or at least substantially or essentially free of a preservative, or alternatively includes not more than a trace amount of a preservative.

Trace amounts of preservatives can include relatively low concentrations or amounts of preservatives in a pharmaceutical composition. In certain embodiments, relatively low concentrations of preservatives include concentrations of about 1 μM or less, or about 1% of the pharmaceutical composition by weight or less or about 1 μg per dosage unit of pharmaceutical composition or less.

In other embodiments, relatively low concentrations of preservatives include concentrations of about 100 nM or less, about 10 nM or less, about 1 nM or less, about 100 pM or less, about 10 pM or less or about 1 pM or less; or about 0.1% or less, or about 0.01% or less, or about 0.001% or less or about 0.0001% or less, each of the pharmaceutical composition by weight.

In other embodiments, relatively low amounts of preservatives in pharmaceutical compositions include pharmaceutical compositions wherein preservatives are provided at about 100 ng or less, about 10 ng or less, about 1 ng or less, about 100 pg or less, about 10 pg or less or about 1 pg or less, each per dosage unit of pharmaceutical composition.

The term “anti-oxidant” for the purposes of the present invention refers to a chemical substance that is added to a pharmaceutical composition to prevent or inhibit the oxidation of molecules that are present in the active component of the composition, such as epinephrine. It is explicitly understood that for the purposes of the present application, anti-oxidants are not considered preservatives. Accordingly, compositions that comprise anti-oxidants are considered preservative-free if they include no other preservative(s).

The term “therapeutically effective amount” is defined as the amount of the compound or pharmaceutical composition that will elicit the biological or medical response of a tissue, system, animal or human, that is being sought by the researcher, medical doctor or other clinician.

The term “pharmaceutically acceptable” when used in reference to a carrier, whether diluent or excipient, refers to substance that is compatible with the other ingredients of the formulation and not deleterious to the recipient thereof.

The terms “administration of a composition” or “administering a composition” is defined to include an act of providing a compound of the invention or pharmaceutical composition to the subject in need of treatment.

### B. Embodiments of the Invention

According to embodiments of the present invention, pharmaceutical compositions are provided for treating or mitigating glaucoma. The compositions include a therapeutically effective quantity of each of at least one first component, at least one second component, at least one third component, and at least one fourth component, as described below in more detail. The components of the composition may form a homogeneous mixture or be in a formed of a dispersed system, such as a colloidal suspension. In some embodiments, the compositions described in the present application, are completely, substantially or essentially free of preservatives and in some further embodiments may contain not more than a trace amount of preservatives, as defined above.

The first component to be used is at least one α-2-adrenergic agonist in an amount of between about 0.1 mass % and about 1.0 mass % of the composition, such as between about 0.1 mass % and about 0.5 mass %, for example about 0.2 mass %. One example of a suitable α-2-adrenergic agonist that may be so used alone or, if desired, in combination with other α-2-adrenergic agonist(s) is brimonidine or pharmaceutically acceptable salts and analogs thereof. Additional acceptable α-2-adrenergic agonist(s) that may be used, each alone, or each in any combination with each other or with brimonidine include clonidine, apraclonidine, dipivefrine, 4-NEMD (i.e., 4-(1-naphthalen-1-ylethyl)-1H-imidazole), talipexole, tiamenidine, agmatine, tizanidine, detomidine, tolonidine, cannabigerol, marsanidine, 7-methylmarsanidine, dexmedetomidine, xylazine, xylometazoline, guanfacine, medetomidine, mivazerol, rilmenidine, fadolmidine, guanabenz, lofexidine, romifidine, methamphetamine, or pharmaceutically acceptable salts and analogs thereof.

The second component to be used is at least one carbonic anhydrase inhibitor in an amount of between about 0.5 mass % and about 2.5 mass % of the composition, such as between about 1.0 mass % and about 2.0 mass %, for example about 2.0 mass %.

One example of a suitable carbonic anhydrase inhibitor that may be so used alone or, if desired, in combination with other carbonic anhydrase inhibitor(s) is dorzolamide, or pharmaceutically acceptable salts and analogs thereof. Additional acceptable carbonic anhydrase inhibitor(s) that may be used, each alone, or each in any combination with each other or with dorzolamide include acetazolamide, methazolamide, brinzolamide, diclofenamide, or pharmaceutically acceptable salts and analogs thereof.

The third component to be used is at least one β-adrenergic antagonist in the amount of between about 0.1 mass % and about 1.0 mass % of the composition, such as between about 0.1 mass % and about 0.5 mass %, for example about 0.5 mass %.

One example of a suitable β-adrenergic antagonist that may be so used alone or, if desired, in combination with other β-adrenergic antagonist(s) is timolol, or pharmaceutically acceptable salts and analogs thereof. Additional acceptable β-adrenergic antagonist(s) that may be used, each alone, or each in any combination with each other or with timolol include propranolol, oxyprenolol, nadolol, levobunolol, sotalol, betaxolol, labetolol, carvedilol, atenolol, carteolol, pindolol, bisoprolol, metoprolol, esmolol, nebivolol, or pharmaceutically acceptable salts and analogs thereof.

The fourth component to be used is at least one prostaglandin analog in the amount of between about 0.001 mass % and about 0.005 mass % of the composition, such as between about 0.001 mass % and about 0.005 mass %, for example about 0.005 mass %.

One example of a suitable prostaglandin analog that may be so used alone or, if desired, in combination with other prostaglandin analog(s) is latanoprost, or pharmaceutically acceptable salts and analogs thereof. Additional acceptable prostaglandin analog(s) that may be used, each alone, or each in any combination with each other or with latanoprost include travoprost, unoprostone, bimatoprost, alprostadil, or pharmaceutically acceptable salts and analogs thereof.

According to further embodiments of the present invention, pharmaceutical compositions for treating or mitigating glaucoma are provided, the compositions comprising a therapeutically effective quantity of only either two or three separate pharmaceutically acceptable components out of the four components described above. It is further specifically provided that when a composition comprises at least one β-adrenergic antagonist, such as timolol, and at least one prostaglandin analog, such as latanoprost, then the composition must also include either at least one α-2-adrenergic agonist or at least one carbonic anhydrase inhibitor. In other words, two-component compositions of just timolol and latanoprost are not envisioned as being within the scope of the invention. The components of the composition may form a homogeneous mixture or be in a form of a dispersed system such as a colloidal suspension.

According to embodiments of the present invention, the pharmaceutical compositions described herein may be formulated as solutions or as colloidal systems (i.e., suspensions or emulsions). When formulated as solutions, the compositions containing all four components in the quantities described above may be dissolved in a suitable solvent to be selected by those having ordinary skill in the art, such as in a purified water suitable for ophthalmic solutions.

According to other embodiments of the invention, when the pharmaceutical compositions described herein are formulated as colloidal emulsions or suspensions, the compositions may further include at least one solubilizing and suspending agent, or a combination thereof, and the colloidal system may be fabricated using such agents according to methods and techniques known to those having ordinary skill in the art.

The first solubilizing and suspending agent may be any of non-ionic polyoxyethlene-polyoxypropylene block copolymers, such as products of Poloxamer® or Pluronic® families, e.g., Poloxamer 407® or Pluronic L64®, in the quantity of between about 0.01 mass % and about 10.0 mass %, such as between about 1.0 mass % and about 8 mass %, for example, about 5.0 mass % of the composition.

The second solubilizing and suspending agent may be a water-soluble derivative of cellulose (e.g., carboxymethyl cellulose, methyl cellulose, hydroxyethyl cellulose, or hydroxypropyl cellulose), non-cross-linked or partially cross-linked polyacrylates, polyoxyethylene sorbitan monolaurates, polyoxyethylene sorbitan monopalmitates, polyoxyethylene sorbitan monostearates, polyoxyethylene sorbitan monooleates (e.g., products of the Tween® or Polysorbate® families, such as Polysorbate 80® which is chemically polyoxyethylene (20) sorbitan monooleate) or combinations thereof.

According to further embodiments, methods for fabricating the above-described pharmaceutical compositions are provided. A one-batch formulation method may be used, where the components of the pharmaceutical formulation can be combined in single container; the components may be added to the container simultaneously or consecutively.

The resulting product may then be adapted for topical administration, i.e., formulated as eye drops according to methods known to those having ordinary skill in the art. The medication prepared as described above may then be prescribed and given to a patient for treating or mitigating glaucoma. Among various kinds of glaucoma that may be treated, one kind of treatment that is particularly envisioned according to embodiments of the present invention is the treatment or mitigation of open angle glaucoma.

It will be understood by those having ordinary skill in the art that the specific dose levels and frequency of administration for any particular patient may be varied and will depend upon a variety of factors including the activity of the specific compound employed, the metabolic stability and length of action of that compound, the age, body weight, general health, gender, diet, and the severity of glaucoma for the particular patient being treated.

In additional embodiments, pharmaceutical kits are provided. The kit includes a sealed container approved for the storage of pharmaceutical compositions, and the above-described pharmaceutical composition. An instruction for the use of the composition and the information about the composition are to be included in the kit.

The following examples are provided to further elucidate the advantages and features of the present invention, but are not intended to limit the scope of the invention. The examples are for illustrative purposes only. USP pharmaceutical grade products were used in preparing the formulations described below.

### EXAMPLE 1

### Preparing a Pharmaceutical Composition No. 1

A pharmaceutical composition was prepared as described below. The following products were used in the amounts and concentrations specified:

(1) about 0.68 g of timolol maleate powder;

(2) about 0.30 g of brimonidine tartarate powder;

(3) about 0.005 g of liquid latanaprost;

(4) about 1.115 g of dorzolamide hydrochloride powder;

(5) about 0.413 g of granular sodium chloride;

(6) about 0.2 mL of 5% aqueous solution of benzalkonium chloride;

(7) about 0.1 g of granular anhydrous citric acid;

(8) about 0.0667 of sodium carboxymethyl cellulose;

(9) a quantity of 10% sodium hydroxide for pH adjustment; and

(10) about 100.0 mL of sterile water (suitable for injections grade).

Sodium chloride, timolol maleate, brimonidine tartarate, latanaprost, and dorzolamide hydrochloride, all in the quantities indicated above, may be combined, mixed without 80% of the water and thoroughly stirred until the solids are completely dissolved, followed by adding citric acid and further stirring. The pH of the solution can then be adjusted to between about 5.6 and 6.6 by adding sodium hydroxide dropwise.

Benzalkonium chloride can then be added and stirred to be dissolved followed by adding the remainder of the water and by slowly adding sodium carboxymethyl cellulose while mixing. The solution can then be filtered through a 0.22 micron filter into an appropriate sterile dispensing container.

### EXAMPLE 2

### Preparing a Pharmaceutical Composition No. 2

A pharmaceutical composition was prepared as described below. The composition was prepared in the same manner as that described in Example 1 except that only three active components (timolol maleate, brimonidine tartarate, and dorzolamide hydrochloride) were used instead of four, as no use of latanaprost is envisioned in the composition of this example.

Accordingly, the following products were in the amounts and concentrations specified to prepare the composition:

(1) about 0.68 g of timolol maleate powder;

(2) about 0.30 g of brimonidine tartarate powder;

(3) about 1.115 g of dorzolamide hydrochloride powder;

(4) about 0.413 g of granular sodium chloride;

(5) about 0.2 mL of 5% aqueous solution of benzalkonium chloride;

(6) about 0.1 g of granular anhydrous citric acid;

(7) about 0.0667 of sodium carboxymethyl cellulose;

(8) a quantity of 10% sodium hydroxide for pH adjustment; and

(9) about 100.0 mL of sterile water (suitable for injections grade).

### EXAMPLE 3

### Preparing a Pharmaceutical Composition No. 3

A pharmaceutical composition was prepared as described below. The following products were used in the amounts and concentrations specified:

(1) about 0.68 g of timolol maleate powder;

(2) about 0.225 g of brimonidine tartarate powder;

(3) about 2.23 g of dorzolamide hydrochloride powder;

(4) about 0.354 g of granular sodium chloride;

(5) about 0.15 g of granular sodium citrate;

(6) about 100.0 mL of Pluronic L64®;

(7) about 100.0 mL of sterile water (suitable for injections grade).

Sodium chloride, sodium citrate, and timolol maleate powders were added to about 90% of slightly heated water (27°-32° C.) and stirred until dissolved, followed by adding brimonidine tartarate powder, stirring until a clear solution was obtained and by turning off heat. A quantity of a 10% sodium hydroxide solution was added to adjust the pH to about 5.7.

Dorzolamide hydrochloride powder was then added and mixed in until dissolved, followed by slowly adding Pluronic L64® in a dropwise manner until dissolved and again adjusting the pH to about 5.7±0.1 using the sodium hydroxide solution. Finally, the remainder of the water was added to bring to final volume. The solution was then filtered through a 0.22 micron filter into a 10 mL sterile dispensing container with 0.2 mL overfill.

### EXAMPLE 4

### Preparing a Pharmaceutical Composition No. 4

A pharmaceutical composition was prepared as described below. The following products were used in the amounts and concentrations specified:

(1) about 0.68 g of timolol maleate powder;

(2) about 0.225 g of brimonidine tartarate powder;

(3) about 0.005 g of latanaprost;

(4) about 2.23 g of dorzolamide hydrochloride powder;

(5) about 0.354 g of granular sodium chloride;

(6) about 0.15 g of granular sodium citrate;

(7) about 50.0 mL of Polysorbate 80®;

(8) about 100.0 mL of Pluronic L64®;

(9) about 100.0 mL of sterile water (suitable for injections grade).

Sodium chloride, sodium citrate, and timolol maleate powders were added to about 90% of slightly heated water (27°-32° C.) and stirred until dissolved, followed by adding brimonidine tartarate powder, stirring until a clear solution was obtained and by turning off heat. A quantity of a 10% sodium hydroxide solution was added to adjust the pH to about 5.7.

Dorzolamide hydrochloride powder was then added and mixed in until dissolved followed by slowly adding Polysorbate 80® and, in a dropwise manner, Pluronic L64® until dissolved. Then, latanoprost was added and after about 45 minutes the solution was stirred to dissolve all the solids. The pH was adjusted to about 5.7±0.1 using the sodium hydroxide solution. Finally, the remainder of the water was added to bring to final volume. The solution was then filtered through a 0.22 micron filter into a 5 mL sterile dispensing container with 0.2 mL overfill.

Although the invention has been described with reference to the above examples, it will be understood that modifications and variations are encompassed within the spirit and scope of the invention. Accordingly, the invention is limited only by the following claims.

What is claimed is:

1. A method for treating or mitigating glaucoma, the method comprising administering to a patient in need thereof a therapeutically effective quantity of a pharmaceutical composition, the composition comprising a therapeutically effective quantity of each of at least one first component, at least one second component, at least one third component, and at least one fourth component, wherein:
(a) the first component comprises α-2-adrenergic agonist selected from the group consisting of brimonidine, clonidine, apraclonidine, dipivefrine, 4-(1-naphthalen-1-ylethyl)-1H-imidazole, talipexole, tiamenidine, agmatine, tizanidine, detomidine, tolonidine, cannabigerol, marsanidine, 7-methylmarsanidine, dexmedetomidine, xylazine, xylometazoline, guanfacine, medetomidine, mivazerol, rilmenidine, fadolmidine, guanabenz, lofexidine, romifidine, methamphetamine, and pharmaceutically acceptable salts and analogs thereof;
(b) the second component comprises a carbonic anhydrase inhibitor selected from the group consisting of dorzolamide, acetazolamide, methazolamide, brinzolamide, diclofenamide, and pharmaceutically acceptable salts and analogs thereof;
(c) the third component comprises a β-adrenergic antagonist selected from the group consisting of timolol, propranolol, oxyprenolol, nadolol, levobunolol, sotalol, betaxolol, labetolol, carvedilol, atenolol, carteolol, pindolol, bisoprolol, metoprolol, esmolol, nebivolol, and pharmaceutically acceptable salts and analogs thereof; and
(d) the fourth component comprises a prostaglandin analog selected from the group consisting of latanoprost, travoprost, unoprostone, bimatoprost, alprostadil, and pharmaceutically acceptable salts and analogs thereof,
wherein the first component, the second component, the third component and the fourth component of the composition form a homogeneous mixture, and wherein the composition is optionally free of preservatives.

2. The method of claim 1, wherein the composition is administered in the form of topical eye drops.

3. The method of claim 1, wherein the first component is brimonidine, the second component is dorzolamide, the third component is timolol, and the fourth component is latanoprost.

4. The method of claim 3, wherein the concentration of brimonidine in the composition is about 0.2 mass %, the concentration of dorzolamide in the composition is about 2.0 mass %, the concentration of timolol in the composition is about 0.5 mass %, and the concentration of latanoprost in the composition is about 0.005 mass %.

5. The method of claim 1, wherein the glaucoma is open-angle glaucoma.

6. The method of claim 1, wherein the composition further comprises a quantity of a first solubilizing and suspending agent selected from the group consisting of at least one non-ionic polyoxyethlene-polyoxypropylene block copolymer.

7. The method of claim 6, wherein the non-ionic polyoxyethlene-polyoxypropylene block copolymer is poly(ethylene glycol)-block-poly(propylene glycol)-block-poly(ethylene glycol).

8. The method of claim 6, wherein the composition further comprises a second solubilizing and suspending agent selected from the group consisting of a water-soluble derivative of cellulose, optionally partially cross-linked polyacrylates, polyoxyethylene sorbitan monolaurates, polyoxyethylene sorbitan monopalmitates, polyoxyethylene sorbitan monostearates, polyoxyethylene sorbitan monooleates, or combinations thereof.

9. The method of claim 8, wherein the second solubilizing and suspending agent is selected from the group consisting of carboxymethyl cellulose, methyl cellulose, hydroxyethyl cellulose, hydroxypropyl cellulose, and polyoxyethylene (20) sorbitan monooleate.

10. A pharmaceutical composition for treating or mitigating glaucoma, the composition comprising a therapeutically effective quantity of each of at least one first component, at least one second component, at least one third component, and at least one fourth component of claim 1.

11. The pharmaceutical composition of claim 10, wherein the composition comprises a colloidal system comprising:
(a) a dispersed phase comprising particles including the composition of claim 10; and
(b) a dispersion medium comprising:
(b1) a first solubilizing and suspending agent selected from the group consisting of at least one non-ionic polyoxyethlene-polyoxypropylene block copolymer;
(b2) a second solubilizing and suspending agent selected from the group consisting of a water-soluble derivative of cellulose, optionally partially cross-linked polyacrylates, polyoxyethylene sorbitan monolaurates, polyoxyethylene sorbitan monopalmitates, polyoxyethylene sorbitan monostearates, polyoxyethylene sorbitan monooleates or combinations thereof; and
(b3) a pharmaceutically acceptable carrier,
wherein the dispersed phase is dispersed within the dispersion medium, with the further proviso that the pharmaceutical composition is an ophthalmic composition that is suitable for delivery via eye drops.

12. The pharmaceutical composition of claim 11, wherein the first solubilizing and suspending agent is poly(ethylene glycol)-block-poly(propylene glycol)-block-poly(ethylene glycol).

13. The pharmaceutical composition of claim 11, wherein the second solubilizing and suspending agent is polyoxyethylene (20) sorbitan monooleate.

14. A method for treating or mitigating glaucoma, the method comprising: administering to a patient in need thereof a therapeutically effective quantity of a pharmaceutical composition, the composition comprising any combination of therapeutically effective quantities of any two or three components selected from the group consisting of the first component, the second component, the third component, and the fourth component of claim 1, wherein the first component, the second component, the third component and the fourth component of the composition form a homogeneous mixture,
with the further proviso that when the composition comprises the third component and the fourth component, the composition also includes the first component or the second component.

15. The method of claim 14, wherein the composition is administered in the form of topical eye drops.

16. The method of claim 14, wherein the first component is brimonidine, the second component is dorzolamide, the third component is timolol, and the fourth component is latanoprost.

17. The method of claim 16, wherein the concentration of brimonidine in the composition is about 0.2 mass %, the concentration of dorzolamide in the composition is about 2.0 mass %, the concentration of timolol in the composition is about 0.5 mass %, and the concentration of latanoprost in the composition is about 0.005 mass %.

18. The method of claim 14, wherein the glaucoma is open-angle glaucoma.

19. The method of claim 14, wherein the composition further comprises a quantity of a first solubilizing and suspending agent selected from the group consisting of at least one non-ionic polyoxyethlene-polyoxypropylene block copolymer.

20. The method of claim 19, wherein the non-ionic polyoxyethlene-polyoxypropylene block copolymer is poly(ethylene glycol)-block-poly(propylene glycol)-block-poly(ethylene glycol).

21. The method of claim 19, wherein the composition further comprises a second solubilizing and suspending agent selected from the group consisting of a water-soluble derivative of cellulose, optionally partially cross-linked polyacrylates, polyoxyethylene sorbitan monolaurates, polyoxyethylene sorbitan monopalmitates, polyoxyethylene sorbitan monostearates, polyoxyethylene sorbitan monooleates, and combinations thereof.

22. The method of claim 21, wherein the second solubilizing and suspending agent is selected from the group consisting of carboxymethyl cellulose, methyl cellulose, hydroxyethyl cellulose, hydroxypropyl cellulose, and polyoxyethylene (20) sorbitan monooleate.
